(19) United States
(12) Patent Application Publication
(10) Pub. No.: US 2026/0201445 A1
(43) Pub. Date: Jul. 16, 2026

(54) BIOMARKERS AND METHODS FOR DETERMINING BRAIN CANCER PROGNOSIS AND TREATMENT

(71) Applicant: University of South Florida, Tampa, FL (US)

(72) Inventors: George Blanck, Tampa, FL (US); Teresa Marie Thomas, Tampa, FL (US)

(21) Appl. No.: 19/449,058

(22) Filed: Jan. 14, 2026

Related U.S. Application Data
(60) Provisional application No. 63/745,105, filed on Jan. 14, 2025.

(51) Int. Cl. C12Q 1/6809; A61K 38/26; A61K 45/06; C07K 16/22

(52) U.S. Cl. CPC C12Q 1/6809; A61K 38/26; A61K 45/06; C07K 16/22; C12Q 2600/118

(57) ABSTRACT

The present disclosure relates to the methods of determining and association of selective biomarkers with overall survival of subjects with low-grade glioma (LGG) or glioblastoma. Further disclosed are methods of diagnosing and treating subjects with low-grade glioma (LGG) or glioblastoma.

### CROSS-REFERENCE TO RELATED APPLICATIONS

This application claims the benefit of priority to U.S. Provisional Patent Application No. 63/745,105, filed on Jan. 14, 2025, the disclosure of which is expressly incorporated by reference herein in its entirety.

### FIELD

Disclosed herein are biomarkers and methods for determining or predicting the survival of patients with low-grade glioma (LGG) or glioblastoma. Further disclosed are methods of diagnosing and treating patients with low-grade glioma (LGG) or glioblastoma.

### BACKGROUND

Gliomas are histologically graded from I to IV. Grade I gliomas are usually benign. Grade II (Low-grade Glioma (LGG) has an average survival period of approximately 7 years. Grade II gliomas can progress into grade III (high-grade gliomas), and eventually grade IV (secondary glioblastoma). Recent research indicates that historically used clinical variables in LGG are inferior prognostic indicators relative to current genetic information, for example, IDH1 mutational status. As LGG is an early stage in the progression of gliomas from low-grade to high grade, there is a need to understand the disease process from this early onset. Pinpointing key molecular features that are associated with improved outcomes will help elucidate the disease progression and serve as prognostic markers in the clinic.

Cancer development and progression are driven by a complex interplay of genetic, epigenetic, and metabolic alterations. Among these, copy number variations have long been recognized as contributors to oncogenesis. Large-scale copy number gains involving well-characterized oncogenes are clearly implicated in tumor initiation and aggressive disease behavior. In contrast, the biological significance of comparatively modest copy number increases has remained poorly defined. Such smaller copy number changes may arise from mechanisms including replication strand slippage or misaligned sister chromatid exchange and are frequently observed across diverse tumor types. Despite their prevalence, the contribution of these subtle genomic alterations to tumor behavior, patient prognosis, and therapeutic response remains insufficiently understood, and robust frameworks for patient stratification based on these events are generally lacking.

Notwithstanding this uncertainty, large-scale cancer genomic analyses have demonstrated a consistent positive correlation between gene copy number and gene expression across multiple cancer types. This relationship suggests that even relatively small copy number changes may have functional consequences by modulating transcript abundance and downstream cellular pathways. However, identifying which biological processes are most sensitive to such copy number-driven effects has proven challenging, particularly in the context of heterogeneous tumor genomes.

Microsatellite instability has emerged as an important indicator of defects in DNA mismatch repair pathways and is widely used to characterize genomic instability in cancer. Advances in sequencing technologies and computational analysis have enabled more refined assessments of microsatellite instability using genome-wide microsatellite comparisons. These approaches provide quantitative measures of instability and have shown concordance with established clinical testing methodologies. Importantly, increasing microsatellite instability scores have been observed to correlate with elevated copy number changes in cancer genomes, suggesting an association between DNA repair defects and copy number-driven genomic remodeling. Emerging analyses further indicate that such copy number changes may preferentially affect genes involved in core metabolic processes.

Cancer cell metabolism is frequently reprogrammed to support rapid proliferation and survival under adverse conditions. A hallmark of this reprogramming is the Warburg effect, characterized by increased glucose uptake and a shift toward glycolysis even in the presence of sufficient oxygen. Dysregulation of glucose utilization pathways is increasingly recognized as a central feature of malignant transformation and tumor progression. Given the established relationship between copy number and gene expression, and the critical role of glycolysis in supporting tumor growth, alterations in the copy number of genes associated with glucose metabolism may have significant clinical implications. However, the extent to which copy number changes affecting metabolic pathways can be systematically leveraged for clinical stratification and outcome prediction has not been fully explored. There remains a need for improved diagnosis and treatment based on the association of how copy number alterations in metabolic genes intersect with genomic instability and tumor metabolism. Addressing this need may enable more refined approaches to patient classification and prognostic assessment, particularly in malignancies characterized by pronounced metabolic reprogramming and genomic heterogeneity.

### SUMMARY

Disclosed herein are methods for determining or predicting the survival of patients with glioma, for example including but not limited to low-grade glioma (LGG) and glioblastoma (GBM) based on the copy number variation and Microsatellite Instability (MSI) score of GLPIR, GPI, AMFR, VEGF, GCG, and ACTA1. Further disclosed are methods of diagnosing and treating subjects with low-grade glioma (LGG) and glioblastoma (GBM) based on the copy number variation and Microsatellite Instability (MSI) score of GLPIR, GPI, AMFR, VEGF, GCG, and ACTA1.

In one example, disclosed herein is a method of determining overall survival in a subject with glioma, comprising:
measuring a glucagon-like peptide-1 receptor (GLPIR) gene or a glucose-6-phosphate isomerase (GPI) gene copy number ratio in a biological sample from a subject relative to a reference control;
determining the GLPIR gene or the GPI gene copy number ratio to an upper quartile or a lower quartile based on a reference glioma population, wherein the glioma comprises glioblastoma (GBM) or lower-grade glioma (LGG); and
determining overall survival in the subject relative to the reference glioma population, wherein the GPI copy number ratio to the upper quartile relates to a decrease in overall survival, and wherein the GLPIR copy number ratio to the upper quartile relates to an increase in overall survival.

In some examples, the biological sample comprises brain tumor tissue, brain tumor cells, biopsy tissue or combination thereof.

In some examples, an increase in the GPI copy number ratio is associated with increased glycolytic activity in tumor cells. In some examples, an increase in the GPI copy number ratio is associated with increased expression of autocrine motility factor produced by GPI, and wherein the autocrine motility factor contributes to tumor progression or poor prognosis.

In one example, disclosed herein is a method of treating glioblastoma (GBM) in a subject, comprising:
determining a glucagon-like peptide-1 receptor (GLPIR) copy number ratio in a biological sample from a subject relative to a reference control;
determining the GLPIR copy number ratio to an upper quartile or a lower quartile based on a reference population; and
administering a GLPIR agonist to the subject with the GLPIR copy number ratio in the upper quartile.

In some examples, the GLPIR agonist is selected from exenatide, liraglutide, semaglutide, dulaglutide, tirzepatide, or a pharmaceutically acceptable analog or derivative thereof.

In some examples, the GLPIR agonist reduces tumor associated edema, intracranial swelling, hypertension, or corticosteroid burden in the subject.

Some examples, further comprise administering a chemotherapy, a radiotherapy, an immunotherapy, or an anti-angiogenic therapy. Some examples, further comprise determining an MSIsensor score in the biological sample and identifying subjects with both upper quartile GLPIR copy number ratios and elevated MSIsensor scores as candidates for combination therapy. Some examples, further comprise administering an immune checkpoint inhibitor to the subject with both GLPIR copy number ratio in the upper quartile and elevated MSIsensor score.

In some examples, the GLPIR copy number ratio to the upper quartile is greater than or equal to a threshold corresponding to 75^{th} percentile of the reference population, and the GLPIR copy number ratio to the lower quartile is less than or equal to a threshold corresponding to 25^{th} percentile of the reference population.

In one example, disclosed herein is a method of treating lower-grade glioma (LGG) in a subject, comprising:
determining a GPI copy number ratio in a biological sample obtained from a subject;
determining the GPI copy number ratio to an upper quartile or a lower quartile based on a reference population; and
administering a GPI antagonist or GPI inhibitor to the subject with the GPI copy number ratio in the upper quartile.

In some examples, the GPI inhibitor comprises a glycolysis inhibitor selected from a group consisting of a phosphofructokinase inhibitor, a hexokinase inhibitor, a glucose transporter inhibitor, and a lactate dehydrogenase inhibitor, or a vascular endothelial growth factor (VEGF) inhibitor selected from a group consisting of bevacizumab, aflibercept, and a tyrosine kinase inhibitor.

In some examples, the GPI inhibitor comprises a combination therapy, wherein the combination therapy comprises a glycolysis targeting therapeutic agent; an autocrine motility factor pathway inhibitor or autocrine motility factor receptor (AMFR) antagonist; and a VEGF blocking agent, wherein the combination is administered to a subject selected based on an upper quartile GPI copy number ratio.

In one example, disclosed herein is a method of determining overall survival in a subject with glioblastoma (GBM), comprising:
measuring a copy number of a glucagon-like peptide-1 receptor (GLPIR) gene in a biological sample from a subject relative to a reference control; and
determining overall survival in the subject relative to the reference control, wherein an increased GLPIR gene copy number relates to an increase in overall survival.

Some examples comprise assessing microsatellite instability in the biological sample, wherein a higher GLPIR gene copy number is associated with a higher MSIsensor score.

Disclosed herein, in one example, is a method for stratifying glioblastoma subjects into prognostic subgroups, comprising:
measuring a GLPIR copy number value in a biological sample from a subject relative to a reference control;
classifying the copy number value into an upper or lower quartile based on predetermined percentile thresholds;
determining an MSIsensor score in the biological sample obtained from the subject;
identifying the subject with a higher GLPIR copy number and MSIsensor score as the upper quartile, wherein the upper quartile exhibits an increase in overall survival; and
identifying the subject with a lower GLPIR copy number and MSIsensor score as the lower quartile, wherein the lower quartile exhibits a decrease in overall survival.

Some examples further comprise determining autocrine motility factor receptor (AMFR) and glucose-6-phosphate isomerase (GPI) copy number value in the biological sample, wherein a higher AMFR and GPI copy number and MSIsensor score relate to an increase in overall survival.

In some examples, the upper quartile comprises GLPIR copy number values greater than or equal to a threshold corresponding to the 75th percentile of the reference population, and the lower quartile comprises GLPIR copy number values less than or equal to a threshold corresponding to the 25th percentile of the reference population.

In some examples, the GLPIR copy number range in the reference population is a ratio of about 0.4 to about 8.3.

Disclosed herein, in one example, is a method of treating glioblastoma in a subject, comprising:
determining a glucagon-like peptide-1 receptor (GLPIR) copy number ratio in a biological sample from a subject relative to a reference control;
determining the GLPIR copy number ratio to an upper quartile or a lower quartile based on a reference population; and
administering a GLPIR agonist to the subject with the GLPIR copy number ratio in the upper quartile.

In some examples, the GLPIR agonist is selected from exenatide, liraglutide, semaglutide, dulaglutide, tirzepatide, or a pharmaceutically acceptable analog or derivative thereof.

In some examples, the administration of the GLPIR agonist reduces tumor-associated edema, intracranial swelling, hypertension, or corticosteroid burden in the subject.

Some examples, further comprise administering a chemotherapy, a radiotherapy, an immunotherapy, or an anti-angiogenic therapy.

Some examples further comprise determining an MSIsensor score in the biological sample and identifying subjects with both upper-quartile GLPIR copy number ratios and elevated MSIsensor scores as candidates for combination therapy.

Some examples, further comprising administering an immune checkpoint inhibitor to the subject with both GLPIR copy number ratio in the upper quartile and elevated MSIsensor score.

In some examples, the biological sample comprises brain tumor tissue, brain tumor cells, biopsy tissue, or a combination thereof.

Disclosed herein, in one example, is a method for determining overall survival in a subject diagnosed with lower-grade glioma (LGG), comprising:
measuring a glucose-6-phosphate isomerase (GPI) copy number ratio in a biological sample from a subject relative to a reference control;
determining the GPI copy number ratio to an upper quartile or a lower quartile based on a reference lower-grade glioma population; and
determining overall survival in the subject relative to the reference lower-grade glioma population, wherein the GPI copy number ratio within the upper quartile relates to a decrease in overall survival.

In some examples, the increased GPI copy number ratio is associated with increased glycolytic activity in tumor cells.

In some examples, the biological sample comprises brain tumor tissue, brain tumor cells, biopsy tissue, or a combination thereof.

In some examples, the elevated GPI copy number ratio is associated with increased expression of autocrine motility factor produced by GPI, wherein the autocrine motility factor contributes to tumor progression or poor prognosis.

Disclosed herein, in one example, is a method of treating lower-grade glioma in a subject, comprising:
determining a GPI copy number ratio in a biological sample obtained from a subject;
determining the GPI copy number ratio to an upper quartile or a lower quartile based on a reference population; and
administering a GPI antagonist or GPI inhibitor to the subject with the GPI copy number ratio in the upper quartile.

In some examples, the GPI inhibitor comprises:
a glycolysis inhibitor selected from the group consisting of a phosphofructokinase inhibitor, a hexokinase inhibitor, a glucose transporter inhibitor, and a lactate dehydrogenase inhibitor, or
a vascular endothelial growth factor (VEGF) inhibitor selected from the group consisting of bevacizumab, aflibercept, and a tyrosine kinase inhibitor.

Disclosed herein, in one example, is a combination therapy for treating lower-grade glioma (LGG), comprising:
a glycolysis targeting therapeutic agent; an autocrine motility factor pathway inhibitor or autocrine motility factor receptor (AMFR) antagonist; and
a VEGF blocking agent, wherein the combination is administered to a subject selected based on an upper quartile GPI copy number ratio.

### BRIEF DESCRIPTION OF THE DRAWINGS

The accompanying figures, which are incorporated in and constitute a part of this specification, illustrate several examples described below.

FIGS. 1A-1C show the overall survival (OS) analyses for the highest quartile vs. the lowest quartile CNs of the GLPIR gene for the TCGA-GBM dataset. FIG. 1A shows an upper 25th percentile CN group (black line), representing the upper 25% of the CNs as determined by tumor to blood sequencing read ratios (Methods). Lower 25th percentile CN group (grey line). Log-rank p-value=0.0337. FIG. 1B shows the disease-specific survival (DSS) analysis for the highest quartile vs. the lowest quartile CNs of the GLPIR gene for the TCGA-GBM dataset. Upper 25th percentile (black line); lower 25th percentile (grey line). Log-rank p-value=0.0366. FIG. 1C shows the progression-free survival (PFS) analysis for the highest quartile vs. the lowest quartile CNs of the GLPIR gene for the TCGA-GBM dataset. Upper 25th percentile (black line); lower 25th percentile (grey line). Log-rank p-value=0.055.

FIGS. 2A-2C show TCGA-GBM GLPIR Pearson's correlation of CNs with MSIsensor score. FIG. 2A shows TCGA-GBM GLPIR copy number on the x-axis with MSISensor score on the y-axis. R=0.211, sample size (N)=329, Pearson's correlation p-value=0.000113. FIG. 2B shows TCGA-GBM AMFR copy number on the x-axis with MSISensor score on the y-axis. R=0.173, N=329, Pearson correlation p-value=0.00161. FIG. 2C shows TCGA-GBM GPI copy number on the x-axis with MSISensor score on the y-axis. The latter Pearson's correlation involves the removal of one outlier data point. R=0.373, N=328, Pearson's correlation P-value <0.00001.

FIGS. 3A-3C show OS analysis for upper quartile vs. lower quartile CNs of GPI for the TCGA-LGG dataset. FIG. 3A shows the upper 25th percentile CN group (grey line), representing the upper 25% of the CNs as determined by tumor to blood sequencing read ratios (Methods). Lower 25th percentile CN group (black line). Log-rank p-value=0.0006. FIG. 3B shows DSS analysis; upper 25th percentile CN group (grey line); lower 25th percentile (black line). Log-rank p-value=0.00066. FIG. 3C shows the PFS analysis; upper 25th percentile CN group (grey line); lower 25th percentile (black line). Log-rank p-value=0.0001.

### DETAILED DESCRIPTION

Disclosed herein are methods for determining or predicting the survival of patients with low-grade glioma (LGG) based on expression levels of GLPIR, AMFR, GCG, GPI, and ACTA1. Further disclosed are methods of diagnosing and treating patients with low-grade glioma (LGG) based on expression levels of GLPIR, AMFR, GCG, GPI, or ACTA1. Also disclosed herein are methods for determining or predicting survival of patients with glioblastoma or neuroblastoma based on expression levels of GLPIR, AMFR, GCG, GPI, and/or ACTA1. Further disclosed are methods of diagnosing and treating patients with glioblastoma or neuroblastoma based on expression levels or copy number variation of GLP1R, AMFR, GCG, GPI, and/or ACTA1. While the Warburg effect is well-known and frequently studied, the molecular features that facilitate increased tumor cell glycolytic activity have yet to be extensively investigated. The current application shows that the amplification of genes encoding proteins related to glucose metabolism could be a mechanism to facilitate increased glycolysis. Thus, a precision-guided copy number variation analysis approach was applied to the GLP1R, AMFR, GCG, GPI, and ACTA1 genes across three different cancer types. Results indicated that higher copy numbers (CNs) of GLP1R in glioblastoma were associated with better patient outcomes, while high CNs of GPI in lower-grade gliomas were associated with worse outcomes. Results also indicated that high microsatellite instability directly correlated with high CNs for most of the above-mentioned genes. These approaches to assessing tumor metabolism-related genes might lead to more accurate measures of patient risk and potential additional treatment options.

In some examples, a method of determining overall survival in a subject diagnosed with glioblastoma comprises measuring a copy number of a glucagon-like peptide 1 receptor gene in a biological sample obtained from the subject relative to a reference control and determining overall survival of the subject relative to the reference control, wherein an increased GLP1R gene copy number is associated with increased overall survival. As used herein, a biological sample includes tumor tissue, resected tumor material, biopsy tissue, tumor-enriched cells, or nucleic acids extracted therefrom. Reference control includes a matched non-tumor sample from the same subject, including blood, germline DNA, or normal tissue, and can be implemented as the tumor-to-blood sequencing read ratio approach used in the paper.

In some examples, the copy number is expressed as a ratio of tumor sequencing reads mapped to the GLP1R locus divided by sequencing reads mapped to the GLP1R locus in the matched blood sample.

In some examples, increased GLP1R copy number comprises a value above a predetermined threshold derived from a reference population, and the reference population comprises a TCGA GBM cohort processed using the same tumor-to-blood read ratio calculation.

In some examples, the GBM cases are partitioned into upper and lower quartiles based on GLP1R tumor to blood read ratios, and the upper quartile is associated with higher survival probability for OS, DSS, and PFS.

As used herein, “variations” include determining overall survival by classifying the subject into a prognostic subgroup, predicting survival probability, assigning risk, selecting a monitoring intensity, or generating a prognosis score, where each determination uses the GLP1R copy number value relative to the reference control and relative to the distribution of values in the reference population.

Intracranial or brain cancer, also known as a malignant brain tumor, is a fast-growing, life-threatening cancer that can invade and destroy brain tissue. Intracranial or brain cancer can be caused by tumors that start in the brain (primary brain tumors) or spread to the brain from other parts of the body (metastatic brain tumors). Intracranial cancers lead to headaches, seizures, difficulty in speaking, paralysis, balance problems or dizziness, vision issues, or hearing issues. Some exemplary intracranial cancers include, but are not limited to, brain metastasis, glioblastoma, meningioma, cerebral arteriovenous malformation, vestibular schwannoma, neuroblastoma, gliosarcoma, or pituitary adenoma. In some examples, the intracranial cancer is glioblastoma, wherein the glioblastoma comprises high-grade glioma (HGG) or low-grade glioma (LGG). As used herein, a “glioma” refers to a tumor that forms in the brain or spinal cord from glial cells, which support and protect nerve cells. Gliomas are the most common primary brain tumors and can be benign or malignant. They can occur in adults and children, and are highly treatable and curable.

The following description of the disclosure is provided as an enabling teaching of the disclosure in its best, currently known aspects. Many modifications and other aspects disclosed herein will come to mind to one skilled in the art to which the disclosed compositions and methods pertain, having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Terminology

Terms used throughout this application are to be construed with ordinary and typical meaning to those of ordinary skill in the art. However, Applicant desires that the following terms be given the particular definition as defined below.

As used herein, the articles “a,” “an,” and “the” mean “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation, and the like. Administration includes self-administration and administration by another.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof, as used herein, are used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

A “composition” is intended to include a combination of an active agent and another compound or composition, inert (for example, a detectable agent or label) or active, such as an adjuvant.

As used herein, the terms “determining,” “measuring,” “assessing,” and “assaying” are used interchangeably and include both quantitative and qualitative determinations.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acid residues, provided the activity of the fragment is not significantly altered or impaired compared to the non-modified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence, or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exists in a hydrogen-bonded, helical array typically associated with, for example, DNA. In addition to the 100% complementary form of double-stranded oligonucleotides, the term “double-stranded,” as used herein, is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double-stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high-scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, “Microsatellite Instability” (MSI) refers to genomic instability characterized by length variation in microsatellite regions resulting from impaired DNA mismatch repair. MSI is measured to detect mismatch repair (MMR) defects. MSIsensor is a computational tool used to determine MSI status within cancer cells and has been determined to be consistent with the top-tier MSI testing methods. MSIsensor scores use modern sequencing techniques, such as massively parallel sequencing, to compare microsatellites. These microsatellites are repeats found in noncoding regions of the genome, often used for DNA variability assessments between individuals. This report demonstrates a positive correlation between increased MSIsensor scores and increased copy numbers (CNs).

The Warburg effect, as used herein, represents the increased uptake of glucose leading to increased anaerobic glycolysis, which is believed to be the reason, in part, for the rapid proliferation of cancer cells. This Warburg effect can be facilitated by several different mechanisms, such as increasing the availability of glycolysis intermediates (glucose-6-phosphate, fructose-6-phosphate, glyceraldehyde-3-phosphate) for the pentose phosphate pathway, which produces ribose sugars and other factors essential for cellular proliferation. We hypothesized that an increase in CNs of glucose utilization genes would be observed in cancer settings and would lead to worse patient outcomes, presumably because of increased expression of these genes and the resulting facilitation of the Warburg effect.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “subject” or “host” refers to any individual who is the target of administration or treatment. The subject can be a vertebrate, for example, a mammal. Thus, the subject can be a human or a veterinary patient. The term “patient” refers to a subject under the treatment of a clinician, e.g., a physician. The subject can be either male or female.

A control sample or a reference sample, as described herein, can be a sample from a healthy subject or sample, a wild-type subject or sample, or from populations thereof. A reference value can be used in place of a control or reference sample, which was previously obtained from a healthy subject or a group of healthy subjects, or a wild-type subject or sample. A control sample or a reference sample can also be a sample with a known amount of a detectable compound or a spiked sample.

The term “tissue” refers to a group or layer of similarly specialized cells that together perform certain special functions. The term “tissue” is intended to include blood, blood preparations such as plasma and serum, bones, joints, muscles, smooth muscles, lung tissues, and organs.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder (e.g., a cancer), or a symptom of a disease or disorder. The terms “treating” and “treatment” can also refer to a reduction in severity and/or frequency of symptoms, elimination of symptoms and/or underlying cause, prevention of the occurrence of symptoms and/or their underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g., cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, “copy number ratio” refers to a normalized measurement of genomic dosage for a gene relative to an expected diploid baseline. In large genomic datasets such as TCGA, copy number variation analysis pipelines do not report absolute, integer copy numbers. Instead, these pipelines generate a ratio calculated by dividing the measured gene signal intensity in a tumor sample by the expected signal intensity for a normal diploid genome. This approach produces a standardized value that allows comparisons across patients, platforms, and sequencing batches.

A copy number ratio of approximately 1.0 corresponds to normal diploid levels of the gene of interest. In this context, a ratio of 1.0 indicates that the measured genomic dosage is consistent with the two copies expected in a typical human genome. A ratio below 1.0 reflects a reduction in genomic dosage and, therefore, indicates deletion or copy loss involving the gene. Such values may arise from hemizygous deletions, deep deletions, or structural losses affecting chromosome segments that contain the gene. A ratio above 1.0 signifies an increase in genomic dosage and therefore indicates amplification of the gene. Ratios above the diploid baseline are typically associated with gain events, including low-level gains or high-level focal amplifications, depending on the magnitude of the ratio.

In some examples, disclosed herein is the TCGA GBM reference population, in which the glucagon-like peptide-1 receptor (GLP1R) copy number ratio spans a range from approximately 0.4 to 8.3. Values toward the lower end of this range reflect deletion or allelic loss, while values toward the upper end reflect marked amplification of the GLP1R gene. These normalized values are used to generate quartile thresholds that divide the reference population distribution into the lower quartile, upper quartile, and intermediate percentiles. The use of normalized copy number ratios enables robust comparisons across clinical cohorts and facilitates stratification of subjects into prognostic subgroups based on gene dosage.

In some examples, numbers expressing quantities of ingredients, properties such as molecular weight, reaction conditions, and so forth, used to describe and claim certain examples of the present disclosure, are to be understood as being modified in some instances by the term “about.” In some examples, the term “about” is used to indicate that a value includes the standard deviation of the mean for the device or method being employed to determine the value. In some examples, the numerical parameters set forth in the written description and attached claims are approximations that can vary depending upon the desired properties sought to be obtained by a particular embodiment. In some examples, the numerical parameters should be construed in light of the number of reported significant digits and by applying ordinary rounding techniques. Notwithstanding that the numerical ranges and parameters setting forth the broad scope of some examples of the present disclosure are approximations, the numerical values set forth in the specific examples are reported as precisely as practicable. The numerical values presented in some examples of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

Throughout this application, various publications are referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned, and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=XN100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides into another in DNA or RNA. Deletion refers to the deletion of one or more amino acids in a protein or the deletion of one or more nucleotides from another in DNA or RNA. Generally, substitutions, insertions, or deletions can be made at any position so long as the required activity is retained. So-called conservative exchanges can be carried out in which the amino acid that is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Exemplary nucleic acids that may be introduced to a host cell include, for example, DNA sequences or genes from another species, or even genes or sequences that originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Methods of down-regulation or silence genes are known in art. For example, expressed protein activity can be downregulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

As would be apparent, the sequencing may be done using a next generation sequencing platform, e.g., Illumina's reversible terminator method, Roche's pyrosequencing method, Life Technologies' sequencing by ligation (the SOLID platform) or Life Technologies' Ion Torrent platform, etc. Examples of such methods are described in the following references: Margulies et al (Nature 2005 437:376-80); Ronaghi et al (Analytical Biochemistry 1996 242:84-9); Shendure (Science 2005 309:1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. In other examples, the sequencing may be done using nanopore sequencing (e.g. as described in Soni et al Clin Chem 53:1996-2001 2007, or as described by Oxford Nanopore Technologies).

A “Reference population” refers to a cohort of cases used to determine distribution-based thresholds and classification cutoffs for copy number ratios and, optionally, MSI-related values. In some examples, the reference population comprises TCGA-GBM cases having paired tumor and blood whole exome sequencing data, for which copy number ratios are computed per case identifier and survival data are available. In some examples, the reference population defines percentile thresholds, including a 25th percentile cutoff and a 75th percentile cutoff, for assigning a subject to a lower quartile group or an upper quartile group based on the subject's copy number ratio. In some examples, the reference population is disease-specific, comprising a glioblastoma reference population for GLP1R stratification and a lower-grade glioma reference population for GPI stratification.

The term “Copy number ratio” refers to a value representing relative gene copy number determined by sequencing read counts in a tumor sample relative to a reference control sample. In some examples, the copy number ratio is calculated for a gene by: obtaining a tumor whole exome sequencing file and a reference control whole exome sequencing file for a given case identifier; generating gene specific file slices spanning the gene coordinates according to a human reference genome assembly; extracting read counts from the tumor file slice and the reference control file slice; calculating a ratio equal to tumor read count divided by reference control read count; or outputting the case identifier and the ratio for downstream stratification. In some examples, the gene coordinates are defined using start and end nucleotide positions according to hg38.

As used herein, the “Upper quartile” refers to the highest quartile of copy number ratios in the reference population distribution, comprising ratios at or above a threshold corresponding to the 75th percentile. As used herein, the “Lower quartile” refers to the lowest quartile of copy number ratios, comprising ratios at or below a threshold corresponding to the 25th percentile. In some examples, for GLP1R in a glioblastoma reference population, the upper quartile comprises ratios of about 1.2 to about 8.3, and the lower quartile comprises ratios of about 0.4 to about 0.91. In some examples, the range of GLP1R copy number ratios in the reference population is about 0.4 to about 8.3.

In some examples, MSI association is evaluated by comparing MSIsensor scores between upper and lower copy number quartiles using a Wilcoxon test or by assessing the correlation between copy number ratio and MSIsensor score across the full dataset using Pearson correlation. In the glioblastoma dataset described in the paper, higher copy number is associated with higher MSIsensor score for GLP1R, AMFR, and GPI by Wilcoxon testing and by Pearson correlation analyses.

In some examples herein, the overall survival relative to a reference control refers to stratifying, predicting, or assigning a prognosis based on whether the subject's copy number ratio falls within a defined distribution relative to the reference population. In some examples, survival distinctions between upper quartile and lower quartile groups are evaluated using Kaplan-Meier analysis and log-rank testing and may be verified using statistical software.

In some examples, the “reference control” comprises a matched non-tumor sample from the same subject, for example, a blood sample, and the copy number value is expressed as a tumor to blood sequencing read ratio for a defined GLP1R genomic region. In some examples, the reference control comprises one or more of: a matched normal tissue sample from the subject; a pooled normal control derived from two or more non-tumor samples; or an in silico reference representing the expected diploid copy number for the region, optionally adjusted for sequencing. In some examples, the reference control is implemented as a reference file that maps each sequencing file to a case identifier and specimen type (tumor or blood), enabling automated pairing of tumor and reference control files for the subject. As used herein, a “reference population” refers to a cohort used to define distribution-based thresholds for stratification of copy number values and, optionally, MSI related values. In some examples, the reference population comprises a set of cases from a curated dataset (for example, TCGA-GBM PanCancer Atlas cases) having paired tumor and reference control sequencing data, where survival endpoints are available, and copy number values are calculated for each case. In some examples, the reference population is used to define percentile thresholds, including the 25th percentile threshold and the 75th percentile threshold, for assigning a subject to a lower quartile group or an upper quartile group.

The term “copy number,” as used herein, refers to the number of genomic copies of a gene or genomic region present in a cell, tissue, or biological sample. Copy number includes normal diploid states, copy number gains, copy number losses, and amplifications. Copy number may be expressed as an absolute value or as a relative value normalized to a reference control. Copy number value or copy number ratio refers to a quantitative value that reflects the relative copy number for a genomic region of interest, optionally expressed as a sequencing read count ratio. To determine the relative or absolute number of copies of a gene in a biological sample. Measuring may be performed using sequencing-based methods, hybridization-based methods, amplification-based methods, or computational analyses thereof. In some examples, measuring comprises determining a ratio of tumor-derived sequencing reads to reference-derived sequencing reads.

In some examples, the copy number ratio for GLP1R is calculated as: copy number ratio=tumor read count for GLP1R region divided by reference control read count for GLP1R region, where read counts are obtained from tumor and reference control file slices spanning the GLP1R genomic coordinates, and the ratio is computed per case identifier.

The term “copy number variation” refers to a structural genomic variation in which a gene or genomic region exhibits a copy number that differs from a reference copy number. CNVs include deletions, duplications, and amplifications and may be germline or somatic in origin.

“GLP1R gene” refers to the gene encoding the glucagon-like peptide 1 receptor, including all exons, introns, untranslated regions, promoter regions, regulatory elements, and genomic variants thereof. The term includes the human GLP1R gene as defined by a human reference genome assembly and encompasses copy number altered forms of the gene.

As used herein, “biological sample” refers to any sample obtained from a subject that contains nucleic acids suitable for genomic analysis. Biological samples include but are not limited to tumor tissue, resected tumor material, biopsy samples, blood, plasma, serum, cerebrospinal fluid, and circulating tumor nucleic acids. As used herein, tumor sample refers to a biological sample obtained from tumor tissue or tumor-derived material from a subject. Tumor samples include primary tumor samples, recurrent tumor samples, and metastatic tumor samples.

As used herein, the term “increased copy number” refers to a copy number that is greater than the copy number of the reference control. Increased copy number includes copy number gain and amplification and may be defined by a threshold, range, or statistical distribution derived from a reference population.

As used herein, “subject” refers to a human individual diagnosed with, suspected of having, or at risk of developing cancer. In some examples, the subject is diagnosed with glioblastoma. The subject may be treatment naive or previously treated.

Glioblastoma refers to a malignant primary brain tumor characterized by high-grade pathology, aggressive growth, and poor prognosis. Glioblastoma includes glioblastoma multiforme and molecularly defined glioblastoma subtypes.

The term “overall survival” (OS) refers to the time from a defined clinical reference point to death from any cause. The clinical reference point may include diagnosis, initiation of treatment, surgical resection, or sample collection. In some examples, overall survival refers to a statistically inferred survival probability derived from comparison to reference cohorts. Determining overall survival may include assigning a subject to a survival category or estimating survival probability without requiring longitudinal follow-up.

The term “survival probability” refers to the likelihood that a subject remains alive over a defined time period, as determined by statistical analysis of clinical and molecular data. As used herein, glioma and glioblastoma refers to different types of brain cancer. “Glioma” is an umbrella term used for primary brain tumors that arise in glial cells. As specialized non-nerve cells in the nervous system, glial cells provide support, insulation and nourishment to nerve cells (neurons), playing a vital role in maintaining the overall health and function of the brain and spinal cord. “Glioblastoma”, refers to an aggressive type of glioma that originates in astrocytes, which are star-shaped glial cells that make up the majority of the cells in the central nervous system (CNS). Astrocytes perform many important functions, including clearing excess neurotransmitters (the body's chemical messengers), stabilizing and regulating the blood-brain barrier and promoting the transmission of signals between the brain and other parts of the central nervous system.

Further, gliomas are present in a varying degree of aggressiveness, reflected in their grade, i.e. grade I-IV per and via clinical data established by the World Health Organization (WHO). “Low-Grade Glioma” or LGG refers to a slower growing glioma brain tumors that will reoccur, progress and likely become more aggressive and more cancerous over time such as astrocytomas, oligodendrogliomas, ependymomas, etc. “High-Grade Gliomas” refer to rapidly growing/progressing brain tumors such as glioblastoma.

### Screening

Also provided are screening methods using the models and cells as described herein. The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of the skills in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of the skills in the art will also be familiar with a variety of search engines to identify commercial sources or desirable compounds and classes of compounds for further testing (see e.g., ZINC database; eMolecules; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts, such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and 0 atoms). Also, drug-like molecules typically have a span (breadth) of between about 8A to about 15A.

In some examples, the anticancer agent is selected from the group consisting of cordycepin, fenretinide, Zyclara, vemurafenib (Zelboraf®), dabrafenib (Tafinlar™), encorafenib (Braftovi™), pembrolizumab (Keytruda), nivolumab (Opdivo), Anthracyclines, Taxanes, 5-fluorouracil (5-FU), Cyclophosphamide (Cytoxan), Carboplatin (Paraplatin), cisplatin, carboplatin, Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), Atezolizumab (Tecentriq), Paclitaxel (Taxol), Albumin-bound paclitaxel (nab-paclitaxel, Abraxane), Docetaxel (Taxotere), Etoposide (VP-16), Pemetrexed (Alimta), Bevacizumab (Avastin), Ramucirumab (Cyramza), ifosfamide (Ifex™), irinotecan (Camptosar™), mitomycin, doxorubicin (Adriamycin), methotrexate, vinblastine (CMV), durvalumab (Imfinzi™), avelumab (Bavencio™), Erdafitinib (Balversa), dacarbazine (DTIC), epirubicin, temozolomide (Temodar™), gemcitabine (Gemzar™), trabectedin (Yondelis™), and Pazopanib (Votrient).

### Methods of Determining Clinical Outcomes

Disclosed herein are methods for predicting clinical outcomes or determining overall survival in subjects diagnosed with glioblastoma by evaluating copy number variation of the GLP1R gene and its relationship with microsatellite instability. The present disclosure further provides examples that differentiate GLP1R from other gene copy number variations that do not confer survival predictive value, and additional examples that integrate combined GLP1R dosage and microsatellite instability profiles to stratify subjects into prognostic groups.

In one example, a method is provided for predicting a survival outcome in a subject with glioblastoma by determining a GLP1R copy number ratio in a biological sample obtained from the subject. As used herein, a copy number ratio refers to the normalized genomic dosage of a gene relative to the expected diploid baseline. In this context, a ratio of approximately 1.0 represents normal diploid levels, a ratio below 1.0 indicates deletion or loss, and a ratio above 1.0 indicates amplification. In the TCGA GBM dataset, which may serve as a reference population distribution, GLP1R copy number ratios range from approximately 0.4 to 8.3. This reference population distribution is processed to calculate quartile thresholds, where the upper quartile is defined as the top 25% of GLP1R copy number ratios, and the lower quartile is defined as the bottom 25%.

In some examples, survival prediction comprises assigning the determined GLP1R copy number ratio to an upper or lower quartile and identifying that subjects with GLP1R ratios in the upper quartile exhibit significantly improved disease-specific survival, overall survival, or progression-free survival relative to subjects in the lower quartile. Kaplan Meier analysis of TCGA GBM cases demonstrates that upper quartile GLP1R copy number values correspond to improved survival probabilities, with statistically significant log-rank values for overall survival, disease specific survival, and progression-free survival. In certain examples, these survival distinctions remain robust after adjusting for clinical covariates through multivariate analysis, thereby confirming that the observed prognostic value of GLP1R dosage is not explained by age, sex, tumor grade, MGMT methylation status, molecular subtype, treatment category, or other conventional clinical risk variables. In some examples, a method of determining overall survival in a subject diagnosed with glioblastoma comprises measuring a copy number of a glucagon-like peptide 1 receptor gene in a biological sample obtained from the subject relative to a reference control and determining overall survival of the subject relative to the reference control, wherein an increased GLP1R gene copy number is associated with increased overall survival. As used herein, a biological sample includes tumor tissue, resected tumor material, biopsy tissue, tumor-enriched cells, or nucleic acids extracted therefrom. Reference control includes a matched non tumor sample from the same subject, including blood, germline DNA, or normal tissue, and can be implemented as the tumor-to-blood sequencing read ratio approach used in the paper.

In some examples, the copy number is expressed as a ratio of tumor sequencing reads mapped to the GLP1R locus divided by sequencing reads mapped to the GLP1R locus in the matched blood sample.

In some examples, the GBM cases are partitioned into upper and lower quartiles based on GLP1R tumor to blood read ratios, and the upper quartile is associated with higher survival probability for OS, DSS, and PFS.

In another example, the methods disclosed herein include determining an MSIsensor score in the same biological sample and correlating the GLP1R copy number ratio with the microsatellite instability measurement. In some examples, elevated GLP1R copy number ratios are significantly associated with higher MSIsensor scores based on Wilcoxon and Pearson analyses, with Wilcoxon p values in the GLP1R cohort approaching 0.00001295 and Pearson correlation p values approximating 0.000095. In some examples, this relationship between GLP1R amplification and microsatellite instability provides an additional molecular context for understanding the biological implications of GLP1R dosage in glioblastoma.

In further examples, multiple genes associated with metabolic and structural regulation may be evaluated for comparison. Copy number ratios of AMFR, GPI, GCG, and ACTA1 are determined and similarly assigned to upper and lower quartiles. Although AMFR, GPI, and ACTA1 exhibit statistically significant correlations between increased copy number ratios and increased MSIsensor scores, none of these genes demonstrate survival associations across any quartile boundary. GCG and ACTA1 further show no meaningful association with microsatellite instability. These examples distinguish GLP1R as a uniquely predictive biomarker among the tested panel of genes, as it alone exhibits both heightened copy number-related microsatellite instability and independent survival predictive capacity.

In some examples, disclosed herein are methods to stratify glioblastoma subjects using a dual parameter approach involving both GLP1R copy number ratio and MSIsensor score. Subjects whose GLP1R values fall in the upper quartile and whose MSIsensor scores are elevated form a prognostic subgroup characterized by improved disease-specific survival. This combined biomarker pattern provides an enhanced means for risk assessment that outperforms either parameter alone.

In some examples, the method further comprises assessing microsatellite instability in the biological sample, wherein higher GLP1R gene copy number is associated with a higher MSIsensor score. As used herein, microsatellite instability refers to genomic instability at microsatellite loci, and MSIsensor score refers to a computed MSI metric derived from sequencing data and used as a quantitative indicator for MSI. In some examples, MSIsensor scores are obtained from an annotated dataset for each case and compared between the upper and lower copy number quartiles using a Wilcoxon test and further evaluated using Pearson correlation across the full dataset.

In some examples, higher GLP1R copy number is associated with higher MSIsensor score using quartile comparison and correlation analysis as described in the paper. Variations include defining elevated MSIsensor score as greater than a cohort median, greater than a cohort mean, greater than the 75th percentile, or greater than a predetermined cutoff selected to maximize separation between survival curves or to maximize correlation with copy number values.

As used herein, the MSIsensor score refers to a quantitative measure of microsatellite instability derived from next generation sequencing data by comparing a tumor sample to a matched normal reference sample from the same subject. In the paired tumor normal configuration described for MSIsensor, microsatellite loci are first defined from the reference genome and then, for each locus with adequate sequencing depth, the distribution of observed repeat lengths in the tumor is statistically compared to the distribution in the matched normal. Loci that show a significant somatic shift in repeat length distribution in the tumor relative to the matched normal are called unstable, and the MSIsensor score is calculated as the percentage of evaluated loci that are called unstable among all loci meeting the coverage requirement, typically expressed as a percent. Accordingly, the score functions as a continuous, genome wide readout of how frequently tumor specific length alterations occur at microsatellite regions, where higher values indicate that a larger fraction of microsatellite loci exhibit tumor specific instability, which is consistent with increased microsatellite instability in that sample.

MSIsensor produces a continuous score that can be used either directly as a quantitative variable or converted into a categorical status such as MSI high versus microsatellite stable using an assay specific cutoff. In the original MSIsensor validation context using TCGA exome data, a cutoff around 3.5 was shown to separate many MSI cases from microsatellite stable cases in a tested cohort, but the specific threshold can vary with sequencing modality, capture design, and locus set, and some workflows using different sequencing panels have applied higher cutoffs such as around 10. As a result, the interpretation is that higher scores reflect more widespread microsatellite instability, while the assignment of MSI high should be defined relative to the validated cutoff for the specific dataset or assay used.

In the current examples, the MSIsensor score values were obtained from a TCGA GBM dataset via cBioPortal and were used as the MSI metric to test associations with gene copy number. Specifically, MSIsensor scores were compared between upper and lower copy number quartiles for each of GLP1R, AMFR, GCG, GPI, and ACTA1 using a Wilcoxon test, and then the association between copy number and MSIsensor score was further evaluated across the full cohort using Pearson correlation. Under this analysis framework, higher copy numbers were reported to be associated with higher MSIsensor scores for GLP1R, AMFR, and GPI, while no significant association was reported for GCG or ACTA1.

With respect to reference control, two related but distinct reference controls are implicated because copy number and MSIsensor reflect different computations. For the copy number analysis described in the paper, the reference control was a matched normal sample, specifically blood, paired to each tumor by case identifier, and the copy number proxy was derived from a tumor to blood sequencing read count ratio computed over gene specific exome slices. Thus, copy number in the tumor was assessed relative to the subject matched blood control for the same genomic regions.

For MSIsensor, the scores used by the authors were retrieved from cBioPortal rather than recomputed from raw sequence files, but the MSIsensor method itself defines microsatellite instability by directly comparing tumor microsatellite length distributions to those from a matched normal reference sample from the same subject, meaning that the underlying reference control for the MSIsensor score is the matched normal sample used to establish the baseline repeat length distributions at each microsatellite locus.

In some examples, the stratification step corresponds to the reported GLP1R ratio ranges where upper quartile ratios are about 1.2 to about 8.3 and lower quartile ratios are about 0.4 to about 0.91. In some examples, the ratio is about 0.35 to about 9.0, about 0.4 to about 9.0, or about 0.4 to about 10, or defining high copy number as at least about 1.1, 1.2, 1.3, 1.5, 2.0, 3.0, 4.0, 5.0, or 8.0, and defining low copy number as less than about 1.0, less than about 0.95, less than about 0.91, or less than about 0.9, where each threshold is selected using the reference population distribution.

In some examples, elevated GPI copy number ratio is associated with increased expression of autocrine motility factor produced by GPI, and autocrine motility factor contributes to tumor progression or poor prognosis. As used herein, autocrine motility factor includes the secreted form of GPI, and contributes to tumor progression includes increased cell motility, invasion, migration, or tumor aggressiveness.

In some examples, the method further comprises determining an autocrine motility factor receptor gene copy number value and a glucose 6 phosphate isomerase gene copy number value in the biological sample, and using one or both of these values with MSIsensor score to refine stratification. As used herein, AMFR refers to a gene encoding the autocrine motility factor receptor, and GPI refers to a gene encoding glucose 6 phosphate isomerase. In some examples, increased AMFR copy number and increased GPI copy number are associated with increased MSIsensor score in the TCGA GBM dataset as shown by quartile comparison and correlation testing.

In some examples, the upper quartile comprises GLP1R copy number values greater than or equal to a threshold corresponding to the 75th percentile of the reference population, and the lower quartile comprises GLP1R copy number values less than or equal to a threshold corresponding to the 25th percentile of the reference population. In this context, threshold corresponding to the 75th percentile means a value above which approximately 25 percent of reference population cases fall, and threshold corresponding to the 25th percentile means a value below which approximately 25 percent of reference population cases fall, where the thresholds are computed from the reference population distribution of tumor to blood read ratios.

In some examples, increased GPI copy number ratio is associated with increased glycolytic activity in tumor cells. As used herein, glycolytic activity includes increased glucose utilization, increased lactate production, increased expression of glycolysis genes, increased uptake on metabolic imaging, or increased enzymatic flux through glycolysis. Increased glycolysis can be measured using one or more biomarkers such as lactate, GLUT expression, or metabolite ratios.

In additional examples, disclosed herein is a diagnostic kit comprising a reagent configured to measure the GLP1R copy number ratio in a biological specimen, a reference chart or computational module defining upper and lower quartile thresholds, and instructions that associate upper quartile GLP1R values with increased survival probability. Such kits may incorporate digital PCR, next generation sequencing, or comparative genomic hybridization platforms, and may further include reagents or software configured to measure and interpret MSIsensor scores.

In some examples, the biological sample comprises one or a combination of brain tumor tissue, brain tumor cells, or biopsy tissue. In some examples, the biological sample comprises a blood sample.

In some examples, the quantifying is carried out to detect gene expression levels. In some examples, the quantifying is carried out by one or a combination of Polymerase Chain Reaction, Real Time-Polymerase Chain Reaction, Real Time Reverse Transcriptase-Polymerase Chain Reaction, Real-time quantitative RT-PCR, Northern blot analysis, in situ hybridization, and probe array.

In some examples, the quantifying is carried out to detect protein expression levels. In some examples, the quantifying is carried out by one or a combination of Western blot, ELISA, flow cytometry, and other methods of detection using antibodies.

Disclosed herein, in certain examples, is the discovery that increased GPI gene copy number in lower-grade glioma correlates with significantly worse patient outcomes and reflects a biologically distinct metabolic phenotype. GPI, also known as glucose 6 phosphate isomerase, phosphohexose isomerase, or phosphoglucose isomerase, encodes a cytoplasmic glycolytic enzyme that catalyzes the conversion of glucose 6 phosphate to fructose 6 phosphate, a rate-shifting step in glycolysis. In some examples, GPI possesses a secreted form referred to as autocrine motility factor, which has been associated with tumor progression, metastatic potential, and poor prognosis across multiple cancers. In certain examples, increased GPI copy number is associated with elevation of both intracellular glycolytic flux and extracellular autocrine motility factor activity.

In some examples, expression of autocrine motility factor is suppressed by inhibitors of vascular endothelial growth factor, and elevated VEGF expression has been linked to increased autocrine motility factor expression in multiple tumor types. In lower-grade glioma, VEGF positive tumors exhibit shortened overall survival and earlier recurrence, and elevated VEGF levels may potentiate autocrine motility factor expression. Based on these relationships, increased GPI copy number may contribute to worsened outcome through both enhanced glycolytic activity and increased autocrine motility factor driven signaling. The present disclosure further recognizes that GPI copy number variation represents only one of two potential mechanisms underlying elevated autocrine motility factor activity in lower-grade glioma, and that increased autocrine motility factor expression may arise from elevated GPI copy number, VEGF induced upregulation, or a combination thereof. These findings provide an opportunity for therapeutic stratification based on GPI dosage, including the selection of subjects for glycolysis targeting agents, autocrine motility factor pathway inhibitors, or VEGF related therapeutics.

In some examples, disclosed herein is a method for stratifying glioblastoma subjects into prognostic subgroups comprises measuring a GLP1R copy number value in a biological sample from a subject relative to a reference control, classifying the copy number value into an upper quartile or a lower quartile based on predetermined percentile thresholds defined from a reference population, determining an MSIsensor score, and identifying the subject as upper quartile when the subject has higher GLP1R copy number and higher MSIsensor score, wherein the upper quartile exhibits increased overall survival, and identifying the subject as lower quartile when the subject has lower GLP1R copy number and lower MSIsensor score, wherein the lower quartile exhibits decreased overall survival. In this context, reference population refers to a set of glioblastoma cases used to derive the percentile thresholds, and predetermined percentile thresholds include a 25th percentile threshold and a 75th percentile threshold that are fixed prior to evaluating an unknown subject. In some embodiments, upper quartile and lower quartile are computed from tumor to blood sequencing read ratios, consistent with the approach described in the paper.

### Methods of Treatment

Disclosed herein, in some examples, are methods for guiding therapeutic decision making in subjects diagnosed with glioblastoma by integrating GLP1R copy number ratio information with clinical considerations and treatment response profiles. In one example, therapeutic selection involves identifying subjects with GLP1R copy number ratios classified within the upper quartile of a reference glioblastoma population, and selecting such subjects for interventions that benefit from improved vascular stability, reduced edema burden, or enhanced control of intracranial pressure.

In one example, a subject identified as an upper quartile GLP1R case is selected for treatment with a GLP1R agonist. GLP1R agonists are known to exert glycemic independent cardiovascular effects including reductions in blood pressure, improvements in endothelial function, and attenuation of pathologic fluid accumulation. These effects can be beneficial in the glioblastoma context, where intratumoral edema, vasogenic swelling, and compromised vascular integrity contribute to morbidity and influence response to several cancer therapies. In some examples, the GLP1R agonist is selected from exenatide, liraglutide, semaglutide, dulaglutide, tirzepatide, or a pharmaceutically acceptable derivative or analog thereof.

In some examples, the treatment selection incorporates the observation that subjects with upper quartile GLP1R copy number ratios exhibit improved disease specific survival. Without being bound by theory, this improvement may derive from a combination of intrinsic tumor biology and systemic physiologic effects linked to GLP1R activation. GLP1R signaling may modulate tumor microenvironment edema, reduce pathologic hypertension, or mitigate the detrimental vascular complications commonly associated with chemotherapeutic, radiation based, or immunotherapy regimens. In this context, the present disclosure provides methods that identify subjects who are more likely to benefit from the ancillary therapeutic effects of GLP1R pathway modulation.

In some examples, a method of treating glioblastoma comprises determining a GLP1R copy number ratio in a biological sample from a subject relative to a reference control, assigning the subject to an upper quartile or a lower quartile based on a reference population, and administering a GLP1R agonist to the subject assigned to the upper quartile. Here, in some examples, treating includes slowing tumor progression, improving a clinical endpoint, reducing edema or steroid use, improving tolerability of adjunct therapies, or improving survival probability. GLP1R agonist refers to an agent that activates GLP1R signaling and includes peptide agonists, peptidomimetics, small molecules, or long acting conjugates.

In one example, the disclosed methods include administering a GLP1R agonist prior to, concurrently with, or following administration of a primary anti cancer therapy such as temozolomide, an immune checkpoint inhibitor, an anti angiogenic agent, a corticosteroid sparing regimen, radiation therapy, or a targeted therapeutic. Subjects whose GLP1R copy number ratios fall within the upper quartile may experience enhanced tolerance to these therapies due to improved fluid balance, reduced edema, and moderated intracranial pressure changes. In some examples, this combination approach may reduce corticosteroid requirements, thereby supporting more robust immune responses during immunotherapy.

In some examples, GLP1R copy number ratio measurements are integrated with MSIsensor scores to refine therapeutic recommendations. Subjects who exhibit both upper quartile GLP1R dosage and elevated microsatellite instability may possess a tumor microenvironment that is more responsive to combined GLP1R agonist and immunomodulatory therapy. Microsatellite instability reflects an elevated mutational burden that may improve the likelihood of response to checkpoint blockade, while GLP1R associated vascular effects may mitigate edema related complications that typically limit aggressive immunotherapy use in glioblastoma.

In certain variations, the therapeutic method includes selecting a glioblastoma subject for combination therapy that includes a GLP1R agonist and an anti VEGF agent. Increased GLP1R dosage may counteract the hypertensive adverse effects associated with some anti VEGF therapies, thereby expanding the therapeutic window for such agents in high risk glioblastoma patients. In these examples, the GLP1R dosage provides a biomarker driven rationale for selecting patients who can safely tolerate anti angiogenic regimens.

In some examples, GLP1R copy number based therapeutic selection is combined with biomarkers unrelated to copy number variation, such as MGMT methylation status, IDH mutation status, TERT promoter mutation status, or radiographic features such as contrast enhancement patterns. These combined biomarkers allow the present methods to generate an integrated profile that directs individualized therapy selection with higher precision.

In certain examples, the therapeutic selection approach includes recommending a reduction or modification of corticosteroid therapy in upper quartile GLP1R subjects due to the enhanced vascular stability predicted by GLP1R amplification. As corticosteroids suppress immune activation, their reduction may improve the efficacy of immunotherapies such as PD1 or CTLA4 blockade. Thus, GLP1R copy number acts as an enabling biomarker for immune based regimens that are otherwise contraindicated due to edema risk.

In some examples, pharmacologic agents that potentiate endogenous GLP1R signaling without directly acting as agonists may be selected. These include inhibitors of DPP4, modulators of GLP1 peptide stability, or agents that enhance GLP1 secretion from enteroendocrine L cells. Such variations fall within the therapeutic strategies contemplated herein.

In further examples, subjects with lower quartile GLP1R copy number ratios may be identified as requiring more intensive edema management or alternative supportive care regimens. The present disclosure provides a biomarker based framework that distinguishes subjects predicted to require enhanced monitoring and more aggressive intracranial pressure control.

In some examples, treatment selection involves adjusting the dosing, scheduling, or sequencing of surgery, chemotherapy, and radiotherapy based on GLP1R dosage profiles. Upper quartile GLP1R subjects may experience improved recovery after surgical resection due to more favorable edema resolution, allowing earlier initiation of adjuvant therapy.

As used herein, an analog refers to a structurally related compound that preserves the core architecture of the parent GLP1R agonist and retains comparable biological activity, whereas a derivative refers to a compound produced by chemical modification of the parent molecule, including salts, esters, prodrugs, conjugates, and other chemically transformed variants.

The term “pharmaceutically acceptable analog or derivative thereof” refers to any chemical modification, structural variant, or closely related compound of a parent GLP1R agonist molecule that retains the ability to activate the GLP1R receptor, produces substantially similar biological activity, and is suitable for administration to a subject without undue toxicity or unacceptable adverse effects. In some examples, such analogs or derivatives include compounds that differ from the parent molecule by one or more substitutions, deletions, insertions, conservative amino acid changes, side chain modifications, cyclization, lipidation, PEGylation, glycosylation, amidation, acylation, or incorporation of non-natural amino acids.

In certain examples, pharmaceutically acceptable analogs include functionally equivalent molecules that bind and activate GLP1R with comparable potency, efficacy, or downstream signaling characteristics. Examples may include long acting GLP1R agonists, enzymatically stabilized GLP1 analogs, DPP4 resistant analogs, or synthetic peptides engineered to enhance receptor half-life or improve pharmacokinetic properties.

In some examples, pharmaceutically acceptable derivatives include salts, esters, prodrugs, solvates, or metabolites of a GLP1R agonist that retain therapeutic activity after in vivo conversion. Such derivatives may be formulated to modify absorption, increase systemic stability, prolong circulation time, or achieve sustained receptor engagement.

Disclosed herein, in one example, are therapeutic methods for treating lower-grade glioma (LGG) by identifying subjects with elevated GPI copy number ratios and administering targeted agents that modulate glycolytic activity or vascular endothelial growth factor mediated signaling. An elevated GPI copy number ratio reflects increased genomic dosage of the GPI gene and is associated with enhanced glycolytic flux, increased metabolic demand, and the potential for greater autocrine motility factor activity in the tumor microenvironment. Subjects classified within the upper quartile of GPI copy number ratios, based on a reference lower-grade glioma population distribution, exhibit significantly worse overall survival, disease specific survival, and progression free survival. These findings provide a rationale for selecting therapeutic agents that specifically counteract the metabolic and microenvironmental consequences of increased GPI gene dosage.

In one example, disclosed herein is a method of treating lower-grade glioma includes administering a GPI inhibitor, wherein the GPI inhibitor comprises a glycolysis inhibitor selected from the group consisting of a phosphofructokinase inhibitor, a hexokinase inhibitor, a glucose transporter inhibitor, and a lactate dehydrogenase inhibitor. Glycolysis inhibitors may reduce the glycolytic capacity of tumor cells with elevated GPI copy number and attenuate the energetic and anabolic advantages conferred by increased GPI expression. In certain examples, inhibition of upstream glycolytic enzymes, such as hexokinase or phosphofructokinase, reduces the availability of downstream intermediates required for rapid tumor growth. In other examples, inhibition of glucose transporters decreases the uptake of extracellular glucose, thereby limiting substrate availability for GPI mediated enzymatic activity. In additional examples, inhibition of lactate dehydrogenase disrupts the conversion of pyruvate to lactate and reduces the acidic microenvironment that promotes tumor invasiveness.

In another example, the GPI inhibitor comprises a vascular endothelial growth factor (VEGF) inhibitor selected from the group consisting of bevacizumab, aflibercept, and tyrosine kinase inhibitors that block VEGF receptor signaling. Increased GPI copy number may elevate the secretion of autocrine motility factor, which can promote tumor progression. Because VEGF has been shown to potentiate autocrine motility factor expression, inhibition of VEGF activity may indirectly suppress autocrine motility factor mediated signaling pathways. In certain examples, VEGF inhibitors reduce tumor vascular permeability, edema formation, and signaling cascades associated with tumor aggressiveness in lower-grade glioma. In some examples, tyrosine kinase inhibitors that target VEGF receptors may reduce VEGF induced upregulation of autocrine motility factor and mitigate the downstream migratory and proliferative effects associated with high GPI dosage.

In further examples, the therapeutic methods disclosed herein include administering the glycolysis inhibitor or VEGF inhibitor prior to, concurrently with, or following a primary treatment regimen comprising surgical resection, radiotherapy, chemotherapy, or immunotherapy. The selection of subjects for these treatments is guided by the determination of GPI copy number ratio, wherein subjects with upper quartile GPI values benefit from therapies that interfere with metabolic dependencies or microenvironmental interactions driven by GPI dosage. These examples provide a precision medicine approach for lower-grade glioma, whereby genomic alterations in metabolic pathways are used to guide rational drug selection.

In some examples, the glycolysis targeting therapeutic agent comprises a glycolysis inhibitor selected from agents that interfere with key enzymatic or transport steps in glucose metabolism. Examples include but are not limited to phosphofructokinase related inhibitors, such as phosphofructokinase-fructose-bisphosphatase 3 (PFKFB3) inhibitors, including 3PO and PFK158, which have been shown to suppress glycolytic flux and tumor growth in preclinical and early clinical studies. In further examples, the glycolysis inhibitor comprises a hexokinase interfering agent, such as 2-deoxy-D-glucose or lonidamine, both of which have been reported to disrupt glucose phosphorylation and downstream glycolytic metabolism in cancer cells. In another example, the glycolysis inhibitor comprises a glucose transporter inhibitor, for example glucose transporter-1 (GLUT1) inhibitors such as WZB117 or BAY 876, which reduce cellular glucose uptake and attenuate glycolysis-dependent tumor phenotypes. In additional examples, the glycolysis inhibitor comprises a lactate dehydrogenase inhibitor, such as FX11, which has been reported to inhibit lactate dehydrogenase A activity, increase oxidative stress, and impair tumor growth in multiple cancer models.

In some examples, the combination therapy further comprises a vascular endothelial growth factor pathway inhibitor. Non-limiting examples of vascular endothelial growth factor blocking agents include but are not limited to bevacizumab, a monoclonal antibody that binds vascular endothelial growth factor A, and aflibercept, a soluble decoy receptor that sequesters vascular endothelial growth factor family ligands. In additional examples, the vascular endothelial growth factor blocking agent comprises a tyrosine kinase inhibitor that targets vascular endothelial growth factor receptors, such as cediranib or axitinib, both of which have been described as inhibitors of vascular endothelial growth factor receptor signaling and have been evaluated in glioma and other solid tumor settings.

In some examples, the combination therapy further comprises an autocrine motility factor pathway inhibitor or an autocrine motility factor receptor antagonist. Autocrine motility factor, also known as phosphoglucose isomerase, has been described as a cytokine-like factor that promotes tumor cell motility and invasion through signaling via its receptor, autocrine motility factor receptor, also referred to as gp78. Reported approaches to inhibiting this pathway include small molecule or metabolite-based inhibitors of phosphoglucose isomerase activity, such as carbohydrate phosphate compounds including erythrose-4-phosphate, as well as antibody-based strategies directed against autocrine motility factor itself. Additional examples include but are not limited to antagonism or targeting of autocrine motility factor receptor using antibody reagents or other binding agents that interfere with autocrine motility factor-autocrine motility factor receptor signaling.

In some examples, the disclosed combination therapy comprises a glycolysis-targeting therapeutic agent, an autocrine motility factor pathway inhibitor or autocrine motility factor receptor antagonist, and a vascular endothelial growth factor blocking agent, wherein the combination is administered to a subject with lower-grade glioma selected based on an elevated or upper quartile glucose-6-phosphate isomerase copy number ratio. Without being bound by theory, such patient selection reflects an association between increased copy number of glycolysis-related genes, altered tumor metabolism, and differential therapeutic responsiveness, thereby providing a rationale for combining metabolic targeting, motility pathway inhibition, and angiogenesis inhibition in a genomically defined lower-grade glioma subpopulation.

In additional examples, the disclosed methods may include determining ancillary biomarkers, such as VEGF expression levels, to further refine the therapeutic strategy. Subjects exhibiting both elevated GPI copy number and elevated VEGF expression may be selected for combination therapy that includes both a glycolytic inhibitor and a VEGF inhibitor to address the dual mechanisms that contribute to poor prognosis. In some examples, directing therapy based on GPI copy number ratio improves treatment outcomes by targeting metabolic vulnerabilities associated with increased glycolytic activity, reducing microenvironmental factors driven by autocrine motility factor, and suppressing VEGF related tumor aggressiveness.

### Administration

Agents and compositions described herein can be administered according to methods described herein in a variety of ways known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, intraparenchymal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 pm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

In some examples, the GLP1R agonist is selected from exenatide, liraglutide, semaglutide, dulaglutide, or tirzepatide, and includes pharmaceutically acceptable salts, solvates, analogs, derivatives, prodrugs, sustained release formulations, and long acting conjugates. Additional examples that can be recited in dependent examples include lixisenatide, albiglutide, efpeglenatide, taspoglutide, beinaglutide, and oxyntomodulin analogs, as examples of GLP1R agonists commonly described in patent literature. Other examples include but are not limited to agonists that act as dual agonists by activating GLP1R and a second receptor, including a GIP receptor, or a GLP1 glucagon dual agonist, provided that GLP1R agonism is present.

In some examples, administration of the GLP1R agonist reduces tumor associated edema, intracranial swelling, hypertension, or corticosteroid burden, wherein the reduction is at least 5 percent, 10 percent, 20 percent, 30 percent, or 50 percent relative to baseline or relative to reference control. As used herein, tumor associated edema includes vasogenic edema detectable by imaging or clinical assessment, intracranial swelling includes increased intracranial pressure or mass effect, hypertension includes elevated systolic or diastolic blood pressure relative to a baseline, and corticosteroid burden includes cumulative dose or duration of steroid therapy required to manage symptoms.

In some examples, the method of treating LGG or brain tumors comprises administering an immune checkpoint inhibitor to the subject with both upper quartile GLP1R copy number ratio and elevated MSIsensor score. Immune checkpoint inhibitor includes PD 1 inhibitors, PD L1 inhibitors, CTLA 4 inhibitors, LAG 3 inhibitors, TIGIT inhibitors, and combinations thereof. Variation includes administering the checkpoint inhibitor concurrently with the GLP1R agonist or sequentially, and selecting subjects using a composite score comprising GLP1R copy number ratio and MSIsensor score.

In some examples, the method further comprises administering a chemotherapy, a radiotherapy, an immunotherapy, or an anti angiogenic therapy. Chemotherapy includes temozolomide, lomustine, carmustine wafer, or combinations thereof. Radiotherapy includes fractionated external beam radiation. Immunotherapy includes immune checkpoint inhibitors, adoptive cell therapies, vaccines, oncolytic viruses, or cytokine therapies. Anti angiogenic therapy includes VEGF pathway inhibitors such as bevacizumab or tyrosine kinase inhibitors.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Therapeutic Methods

Also provided is a process of treating, preventing, or reversing cancer (e.g., glioblastoma) in a subject in need of administration of a therapeutically effective amount of an agent, so as to substantially inhibit cancer, slow the progress of cancer, or limit the development of cancer.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

In some examples, the GPI inhibitor comprises a glycolysis inhibitor selected from a phosphofructokinase inhibitor, a hexokinase inhibitor, a glucose transporter inhibitor, and a lactate dehydrogenase inhibitor, or a VEGF inhibitor selected from bevacizumab, aflibercept, and a tyrosine kinase inhibitor. As used herein, phosphofructokinase inhibitor includes PFK or PFKFB inhibitors, hexokinase inhibitor includes HK2 pathway inhibitors, glucose transporter inhibitor includes inhibitors of GLUT1 or GLUT3 transport, and lactate dehydrogenase inhibitor includes LDHA inhibitors or inhibitors that reduce lactate production. Examples that can be recited in dependent examples include 2 deoxyglucose, lonidamine, dichloroacetate, 3 bromopyruvate, WZB117, BAY 876, and FX11, as non limiting examples of metabolic pathway inhibitors.

In some examples, a combination therapy for treating lower-grade glioma comprises a glycolysis targeting therapeutic agent, an autocrine motility factor pathway inhibitor or an AMFR antagonist, and a VEGF blocking agent, wherein the combination is administered to a subject selected based on an upper quartile GPI copy number ratio. As used herein, AMFR antagonist includes an antibody, peptide, small molecule, or nucleic acid that reduces AMFR activity, reduces AMFR expression, or blocks AMF binding. VEGF blocking agent includes an antibody, soluble receptor, aptamer, or small molecule inhibitor that inhibits VEGF signaling.

Generally, a safe and effective amount of an agent is, for example, an amount that would cause the desired therapeutic effect in a subject while minimizing undesired side effects. In various examples, an effective amount of an agent described herein can substantially inhibit cancer, slow the progress of cancer, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit cancer, slow the progress of cancer, or limit the development of cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an agent can occur as a single event or over a time course of treatment. For example, an agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer. An agent can be administered simultaneously or sequentially with another agent, such as an antibiotic, an anti-inflammatory, or another agent. For example, an agent can be administered simultaneously with another agent, such as an antibiotic or an anti-inflammatory. Simultaneous administration can occur through the administration of separate compositions, each containing one or more of agents, anti-cancer agent, antibiotic, an anti-inflammatory, or another agent. Simultaneous administration can occur through administration of one composition containing two or more agents, anti-cancer agent, antibiotic, anti-inflammatory, or another agent. An agent can be administered sequentially with an anti-cancer agent, an antibiotic, an anti-inflammatory, or another agent. For example, an agent can be administered before or after administration of an anti-cancer agent, an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22 (3): 659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models is also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of the therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide the subject other therapies known to be efficacious for treatment of the disease, disorder, or condition.

Additional advantages will be set forth in part in the following description and in part will be obvious from the description or may be learned by practicing the examples described below. The advantages described below will be realized and attained by the elements and combinations pointed out in the appended claims. It is to be understood that the foregoing general description and the following detailed description are exemplary and explanatory only and are not restrictive.

### EXAMPLES

The following examples are set forth below to illustrate the compounds, systems, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1 Shows GLP1R CNV and TCGA-GBM Survival Distinctions

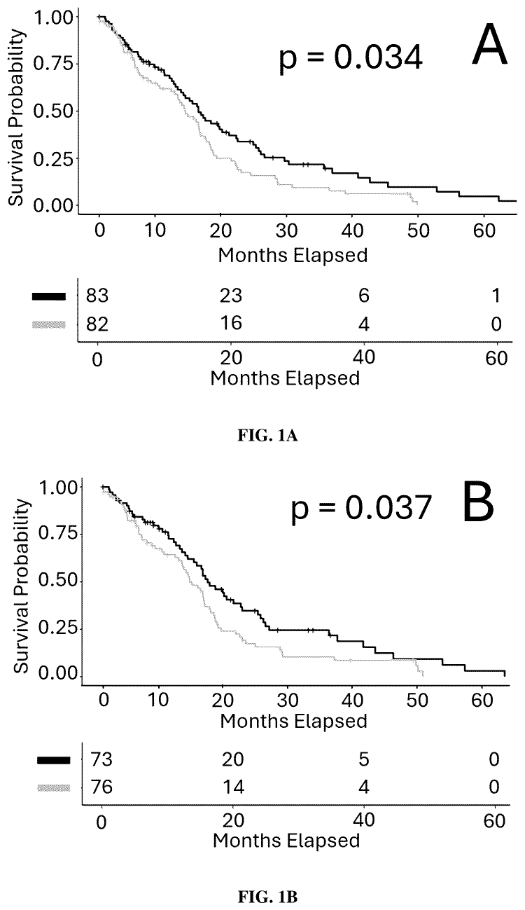

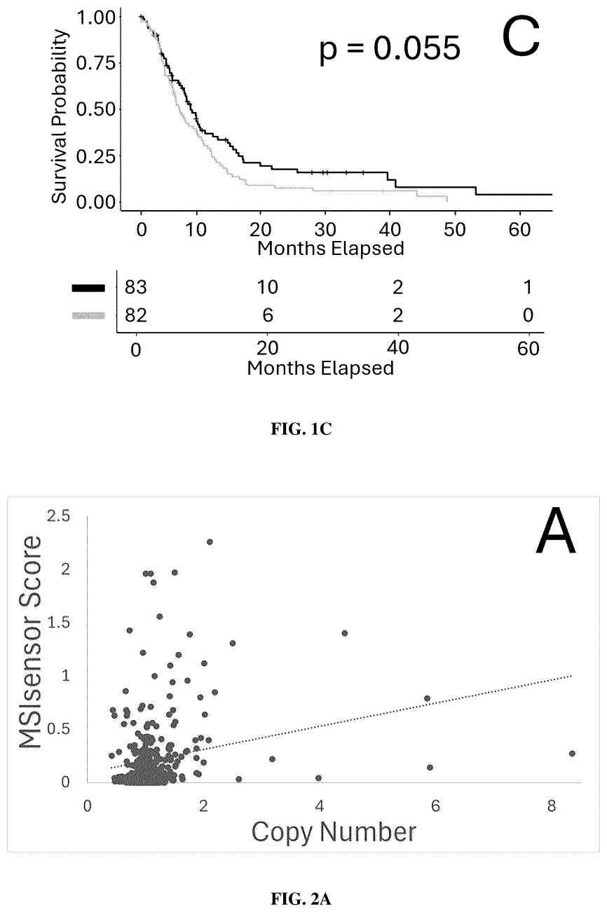

To determine whether copy number variation of the GLP1R gene represents a survival distinction in glioblastoma, a precision guided copy number variation assessment approach is applied, as described in the Methods section. Using this approach, TCGA glioblastoma cases are stratified into upper and lower copy number quartiles based on GLP1R tumor to blood sequencing read ratios, with upper quartile ratios ranging from 1.2 to 8.3 and lower quartile ratios ranging from 0.4 to 0.91. Kaplan Meier analyses show that cases in the upper copy number quartile exhibit a higher survival probability across all evaluated survival parameters, including overall survival, disease specific survival, and progression free survival, as illustrated in FIG. 1A, FIG. 1B, and FIG. 1C.

Four additional genes are subsequently evaluated for copy number variation and survival associations, including three genes related to glucose metabolism and one encoding actin alpha 1, specifically AMFR, GPI, GCG, and ACTA1. For each of these genes, the analyses show no association between copy number variation and survival probability for any of the survival parameters assessed in the GLP1R analysis. In considering the absence of survival associations, it is notable that all evaluated genes display copy number ranges comparable to that of GLP1R. For GLP1R, the copy number ratio ranges from 0.4 to 8.3. By comparison, ACTA1 exhibits a copy number ratio range from 0.05 to 12.3, and GPI exhibits a copy number ratio range from 0.2 to 5.7, as representative examples.

### Example 2 Shows the TCGA-GBM GLP1R Multivariate Analysis

To determine whether the copy number distinctions described above correlate with other common clinical variables, and to address potential confounding factors, a multivariate analysis is conducted. The results indicate that the disease specific survival distinction between the upper and lower copy number quartiles is maintained, with no evidence of correlation with multiple other common clinical variables, as summarized in Table 1.

Table 1 shows a multivariate analysis using mutation Count, radiation therapy status, buffa hypoxia score, and copy numbers (CNs) as variables for disease specific survival. CNV results were from the GLP1R analysis with the TCGA-GBM dataset; HR=hazard ratio; CI=confidence interval.

| Characteristic | HR¹ | 95% CI¹ | p-value |
|---|---|---|---|
| CN | | | 0.024 |
| Bottom Quartile | — | — | |
| Top Quartile | 0.33 | 0.13, 0.88 | |
| Mutation Count | 0.97 | 0.94, 1.00 | 0.033 |
| Radiation Therapy | | | <0.001 |
| No | — | — | |
| Yes | 0.01 | 0.00, 0.10 | |
| Buffa Hypoxia Score | 1.06 | 1.03, 1.09 | <0.001 |

### Example 3 Shows Correlation of MSIsensor Scores and TCGA GBM Copy Numbers

MSIsensor scores for the upper and lower copy number quartiles of the five genes evaluated above, namely GLP1R, AMFR, GCG, GPI, and ACTA1, are obtained and compared to assess whether differences in MSIsensor scores exist between the quartiles. Higher copy numbers are associated with higher MSIsensor scores for three genes, specifically GLP1R, AMFR, and GPI, as shown in Table 2. No significant associations between copy number and MSIsensor scores are observed for the GCG or ACTA1 genes.

Table 2 shows MSIsensor score directly correlates with the upper quartile of CNs for each tested gene. Wilcoxon test p-values for MSIsensor score and CNs associated with the 5 tested genes.

| | | TCGA-LGG | |
|---|---|---|---|
| | | (MSIsensor score | |
| | | does not correlate | |
| Gene | TCGA-GBM | with higher CNs) | TCGA-PAAD |
| GLP1R | 0.00001295 | 0.38 | 0.000000153 |
| AMFR | 0.004481 | 0.0658 | 0.00000000019 |
| GCG | 0.484 | 0.129 | <10\hat{ }−10 |
| GPI | 0.0452 | 0.27 | 0.00000000345 |
| ACTA1 | 0.642 | 0.728 | 0.0000928 |

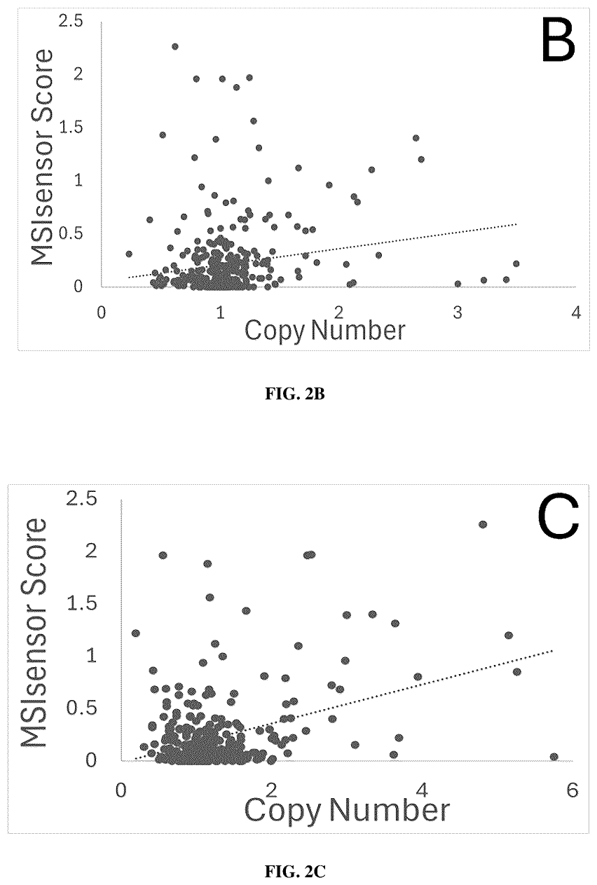

To further evaluate the relationship between copy number and MSIsensor scores, a Pearson correlation analysis is performed using the complete data set. The results are consistent with the quartile based analysis and confirm significant associations between increased copy number and increased MSIsensor scores for GLP1R, AMFR, and GPI, as presented in Table 3 and FIG. 2A, FIG. 2B, and FIG. 2C. No significant Pearson correlations between copy number and MSIsensor scores are identified for the GCG or ACTA1 genes.

Table 3 shows MSIsensor scores directly correlate with the CNs of the five tested genes from TCGA-GBM dataset, using a Pearson's correlation analyses. Pearson's correlation (0.05 significance level) between MSIsensor scores and CNs for TCGA-GBM.

| Gene | Pearson's p-Value |
|---|---|
| GLP1R | 0.000095 |
| AMFR | 0.000113 |
| GCG | 0.3825 |
| GPI | <0.00001 |
| ACTA1 | 0.3972 |

### Example 4 Shows the TCGA-LGG Cases Lack a Correlation of CNs and MSIsensor Scores

To evaluate the specificity of the above findings for glioblastoma and to determine whether a correlation exists between copy number and MSIsensor scores in TCGA low-grade glioma cases, the same upper and lower quartile assessment approach described in the Methods section is applied to the TCGA LGG dataset. The results do not indicate any correlation between copy number assessments and MSIsensor scores for GPI, GLP1R, AMFR, GCG, or ACTA1, as summarized in Table 2. In addition, no associations are observed between copy number and any survival parameters, including overall survival, disease specific survival, or progression free survival.

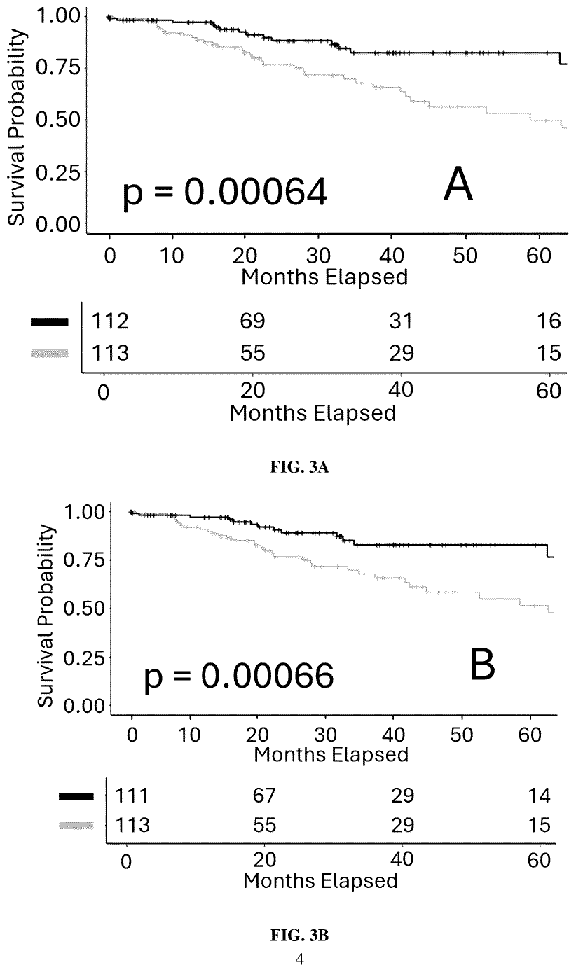

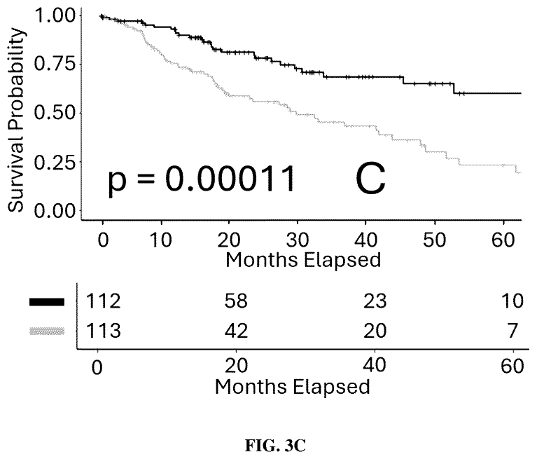

An exception is observed for GPI, for which higher copy numbers are associated with worse survival probabilities for overall survival, disease specific survival, and progression free survival, as shown in FIG. 3A, FIG. 3B, and FIG. 3C. As noted above in the analysis of glioblastoma, all genes that do not show an association between copy number and survival exhibit copy number ranges comparable to that observed for GPI.

### Example 5 Shows the Correlation of MSIsensor and Copy Number Assessments for TCGA-PAAD Cases in the Absence of Outcome Correlations

MSIsensor scores for the upper and lower copy number quartiles of the five genes evaluated above, namely GLP1R, AMFR, GCG, GPI, and ACTA1, are obtained and compared to determine whether differences in MSIsensor scores exist between the quartiles in the TCGA pancreatic adenocarcinoma dataset. For each of the five genes, a difference is observed between the upper and lower quartiles, with the upper copy number quartile exhibiting higher MSIsensor scores, as summarized in Table 1.

To further evaluate this relationship, a Pearson correlation analysis is performed using the full TCGA PAAD dataset of copy number and MSIsensor scores for each gene. The results are consistent with the quartile-based analysis and demonstrate significant associations between higher copy number and higher MSIsensor scores for all five genes, as shown in Table 4. Despite these associations, no significant survival distinctions are identified between the upper and lower copy number quartiles for any of the evaluated genes across any available survival parameters, including overall survival, progression free survival, and disease specific survival.

Table 4 shows the Pearson's correlation p-values of CNs and MSIsensor score for the 5 tested genes in TCGA-PAAD dataset. Pearson correlation (0.05 significance level) between copy number and MSIsensor score for TCGA-PAAD genes.

| Gene | Pearson p-value |
|---|---|
| GLP1R | 0.007269 |
| AMFR | 0.0002 |
| GCG | 0.000026 |
| GPI | 0.000117 |
| ACTA1 | 0.0502 |

### DISCUSSION

The TCGA-GBM GLP1R copy number assessment indicates that higher copy numbers are associated with improved clinical outcomes. This finding is unexpected in light of that increased GLP1R activity would promote greater glucose uptake by cancer cells, thereby supporting glycolysis in a manner consistent with the well accepted Warburg effect. Potential explanations for this unexpected observation are discussed below. In contrast, the TCGA-LGG GPI assessment aligns with the hypothesis that higher GPI copy numbers are associated with worse survival probabilities. This observation is consistent with the concept that increased GPI dosage reflects enhanced glycolytic activity in lower-grade glioma cells. Finally, the TCGA-PAAD assessments demonstrate a lack of correlation between survival probabilities and elevated copy numbers for genes encoding proteins that facilitate tumor cell glycolysis. In pancreatic adenocarcinoma, these copy number changes likely represent one of several molecular mechanisms that support glycolysis, which appears to be a fundamental requirement for disease development. As a result, reliance on one glycolytic mechanism versus another does not appear to stratify patient outcomes in this cancer type.

In the TCGA-GBM dataset, MSIsensor scores are higher in cases with elevated copy numbers for three of the genes evaluated. Microsatellite instability is commonly used as an indicator of mismatch repair defects, and the MSIsensor approach is recognized as being consistent with established microsatellite instability testing methodologies. Accordingly, these results demonstrate, for the first time, a positive correlation between increased MSIsensor scores and increased copy numbers, particularly when copy number is assessed using a precision guided detection method.

In the TCGA-LGG dataset, no correlations are observed between MSIsensor scores and copy numbers for any of the genes analyzed. This result suggests that low-grade glioma may involve repair defects that give rise to copy number variation but are not captured by MSIsensor based measurements. In the TCGA PAAD dataset, the findings are generally consistent with those observed in TCGA GBM, in that the highest copy number quartiles are associated with elevated MSIsensor scores for all five genes evaluated. Pearson correlation analyses further validate these associations in TCGA PAAD and confirm the analogous correlation between MSIsensor scores and copy numbers observed in TCGA GBM.

GLP1R is activated by the glucagon like peptide 1 hormone, which is secreted by intestinal L cells following nutrient intake. Stimulation of GLP1R potentiates multiple metabolic effects, including enhanced insulin secretion, stimulation of pancreatic beta cell proliferation with reduced apoptotic activity, suppression of circulating glucagon levels, increased insulin expression, and delayed gastric emptying. Therapeutic strategies targeting GLP1R stimulation have gained significant attention in recent years due to their ability to promote satiety and induce hypoglycemic effects, thereby supporting the management of obesity and type 2 diabetes.

Beyond these established metabolic effects, GLP1R stimulation has also been associated with cardiovascular benefits across multiple experimental models. GLP1R agonists exhibit long term antihypertensive effects in non diabetic settings, indicating that blood pressure reduction is at least partially independent of glycemic control. Blood pressure modulation is clinically relevant for many cancer treatments, particularly with respect to reducing edema and tissue swelling. At present, edema associated with immunotherapy is often managed using immunosuppressive steroids. However, in mouse models of glioblastoma, the antihypertensive agent losartan reduces edema and enhances antitumor immune responses. Uncontrolled hypertension is associated with increased morbidity and mortality across chemotherapy, radiotherapy, and immunotherapy treatment paradigms. In this study, increased GLP1R copy numbers are associated with improved survival probabilities in patients with glioblastoma. It is hypothesized that glycemic independent blood pressure reduction and cardiovascular protective effects mediated by GLP1R signaling, leading to decreased edema and swelling, may contribute to these observed survival benefits.

In the TCGA-LGG dataset, increased GPI copy numbers are associated with worse patient outcomes. This observation is consistent with the hypothesis that increased copy numbers of glucose utilization genes are linked to poorer prognosis due to enhanced glycolytic activity in tumor cells. GPI, also known as glucose 6 phosphate isomerase, phosphohexose isomerase, or phosphoglucose isomerase, is a cytoplasmic glycolytic enzyme that catalyzes the interconversion of glucose 6 phosphate and fructose 6 phosphate. GPI also exists in a secreted form known as autocrine motility factor, which has been associated with disease progression and poor prognosis in breast cancer. Expression of GPI or autocrine motility factor is suppressed by vascular endothelial growth factor inhibitors, suggesting that activation of the vascular endothelial growth factor signaling cascade may enhance GPI or autocrine motility factor expression. Vascular endothelial growth factor is a widely used cancer biomarker, and its relationship with GPI or autocrine motility factor warrants further investigation. In patients with low-grade astrocytoma and glioblastoma, vascular endothelial growth factor positive tumors are associated with significantly shorter overall survival. In low-grade astrocytoma, vascular endothelial growth factor overexpression is also linked to earlier tumor recurrence. These observations suggest that vascular endothelial growth factor mediated signaling may potentiate increased GPI or autocrine motility factor expression. Based on the present results, an alternative or additional mechanism contributing to elevated GPI or autocrine motility factor levels in low-grade glioma is increased GPI copy number, which is indeed associated with worse clinical outcomes. It is likely that increased expression of GPI or autocrine motility factor reflects a combination of these mechanisms.

The analyses described above are limited to clinical data available through cbioportal.org. A prospective study designed to interrogate related parameters could incorporate clinical variables with closer mechanistic relevance to the molecular findings. Such variables may include blood glucose levels, hemoglobin A1C measurements, or diabetic status, to assess correlations between systemic glucose regulation and cancer development or severity. In addition, the present approach evaluates copy numbers of the AMFR gene, which encodes the receptor for autocrine motility factor, rather than directly assessing extracellular levels of the autocrine motility factor protein. Measurement of microenvironment specific autocrine motility factor levels may yield different insights into its role in cancer progression. Given the increasing clinical use of GLP1R agonists, their potential relevance in the treatment of glioblastoma represents a promising avenue.

### Methods

Accessing exome sequencing files-Whole exome sequencing genomic files for The Cancer Genome Atlas glioblastoma multiforme, lower-grade glioma, and pancreatic adenocarcinoma datasets are accessed under dbGaP project phs000178 through National Institutes of Health Database of Genotypes and Phenotypes project approval number 6300. The files are obtained using the Genomic Data Commons download tool.

Precision guided copy number assessments-Precision guided copy number variation assessments are performed using a file manifest that represents all whole exome sequencing files for each cancer dataset. The file manifests are generated using the Genomic Data Commons download tool. A reference file containing the file name, corresponding case identifier, and tumor or blood designation is used to define the sample type for each file in the manifest. Gene specific whole exome sequencing file slices for copy number assessment are obtained from each file in BAM format using the Genomic Data Commons database. The genomic coordinates for each gene are defined by the start and end nucleotide positions according to the hg38 human reference genome, as indicated by the UCSC Human Genome Browser. Following download, the file slices are renamed to reflect tumor or blood origin and the associated case identifiers using the original manifest file. A matching procedure is then applied to pair each tumor file slice with its corresponding blood file slice for each unique case identifier. Sequencing reads from each file slice are extracted into Excel format using SAMtools functionality. For each case, a tumor to blood sequencing read count ratio is calculated.

Evaluating survival probabilities-Survival data for the TCGA glioblastoma multiforme, lower-grade glioma, and pancreatic adenocarcinoma datasets are obtained from cbioportal.org using PanCancer Atlas data. Survival distinctions are evaluated using the web based analysis tools available on cbioportal.org and are independently verified using R-Studio, which is also used to generate the figures included in the current application.

Assessing MSIsensor score data-MSIsensor score data are obtained from cbioportal.org and are used to compare upper and lower copy number quartiles using Wilcoxon statistical analysis, as described in the Results section. These findings are subsequently verified and extended through Pearson correlation analysis using MSIsensor scores from cbioportal.org and the complete set of copy number values derived from the copy number variation assessments.

Multivariate analysis-Multivariate analysis is performed using R-Studio following established analytical procedures. Copy number variation results from the TCGA glioblastoma multiforme GLP1R analysis and radiation therapy status are treated as categorical variables, while tumor mutation burden and hypoxia score are treated as continuous variables.

The disclosed subject matter includes examples in which exactly one member of the group is present in, employed in, or otherwise relevant to a given product or process. The disclosed subject matter includes examples in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or examples of the invention, is/are referred to as comprising particular elements and/or features, certain examples of the invention or examples of the invention consist, or consist essentially of, such elements and/or features. For purposes of simplicity, those examples have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different examples of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular example of the present invention that falls within the prior art may be explicitly excluded from any one or more of the claims. Because such examples are deemed to be known to one of ordinary skill in the art, they may be excluded even if the exclusion is not set forth explicitly herein. Any particular example of the invention can be excluded from any claim, for any reason, whether or not related to the existence of prior art.

Those skilled in the art will recognize or be able to ascertain using no more than routine experimentation many equivalents to the specific examples described herein. The scope of the present examples described herein is not intended to be limited to the above detailed description, but rather is as set forth in the appended claims. Those of ordinary skill in the art will appreciate that various changes and modifications to this description may be made without departing from the spirit or scope of the present invention, as defined in the following claims.

What is claimed is:

1. A method of determining overall survival in a subject with glioma, comprising:
measuring a glucagon-like peptide-1 receptor (GLP1R) gene or a glucose-6-phosphate isomerase (GPI) gene copy number ratio in a biological sample from a subject relative to a reference control;
determining the GLP1R gene or the GPI gene copy number ratio to an upper quartile or a lower quartile based on a reference glioma population, wherein the glioma comprises glioblastoma (GBM) or lower-grade glioma (LGG); and
determining overall survival in the subject relative to the reference glioma population, wherein the GPI copy number ratio to the upper quartile relates to a decrease in overall survival, and wherein the GLP1R copy number ratio to the upper quartile relates to an increase in overall survival.

2. The method of claim 1, wherein the biological sample comprises brain tumor tissue, brain tumor cells, biopsy tissue or combination thereof.

3. The method of claim 1, wherein an increase in the GPI copy number ratio is associated with increased glycolytic activity in tumor cells.

4. The method of claim 1, wherein an increase in the GPI copy number ratio is associated with increased expression of autocrine motility factor produced by GPI, and wherein the autocrine motility factor contributes to tumor progression or poor prognosis.

5. A method of treating glioblastoma (GBM) in a subject, comprising:
determining a glucagon-like peptide-1 receptor (GLP1R) copy number ratio in a biological sample from a subject relative to a reference control;
determining the GLP1R copy number ratio to an upper quartile or a lower quartile based on a reference population; and
administering a GLP1R agonist to the subject with the GLP1R copy number ratio in the upper quartile.

6. The method of claim 5, wherein the GLP1R agonist is selected from exenatide, liraglutide, semaglutide, dulaglutide, tirzepatide, or a pharmaceutically acceptable analog or derivative thereof.

7. The method of claim 5, wherein the GLP1R agonist reduces tumor associated edema, intracranial swelling, hypertension, or corticosteroid burden in the subject.

8. The method of claim 5, further comprising administering a chemotherapy, a radiotherapy, an immunotherapy, or an anti-angiogenic therapy.

9. The method of claim 5, further comprising determining an MSIsensor score in the biological sample and identifying subjects with both upper quartile GLP1R copy number ratios and elevated MSIsensor scores as candidates for combination therapy.

10. The method of claim 9, further comprising administering an immune checkpoint inhibitor to the subject with both GLP1R copy number ratio in the upper quartile and elevated MSIsensor score.

11. The method of claim 5, wherein the GLP1R copy number ratio to the upper quartile is greater than or equal to a threshold corresponding to 75th percentile of the reference population, and the GLP1R copy number ratio to the lower quartile is less than or equal to a threshold corresponding to 25th percentile of the reference population.

12. A method of treating lower-grade glioma (LGG) in a subject, comprising:
determining a GPI copy number ratio in a biological sample obtained from a subject;
determining the GPI copy number ratio to an upper quartile or a lower quartile based on a reference population; and
administering a GPI antagonist or GPI inhibitor to the subject with the GPI copy number ratio in the upper quartile.

13. The method of claim 12, wherein the GPI inhibitor comprises a glycolysis inhibitor selected from a group consisting of a phosphofructokinase inhibitor, a hexokinase inhibitor, a glucose transporter inhibitor, and a lactate dehydrogenase inhibitor, or a vascular endothelial growth factor (VEGF) inhibitor selected from a group consisting of bevacizumab, aflibercept, and a tyrosine kinase inhibitor.

14. The method of claim 12, wherein the GPI inhibitor comprises a combination therapy, wherein the combination therapy comprises a glycolysis targeting therapeutic agent; an autocrine motility factor pathway inhibitor or autocrine motility factor receptor (AMFR) antagonist; and a VEGF blocking agent, wherein the combination is administered to a subject selected based on an upper quartile GPI copy number ratio.
